SEQUENCE LISTING (2 sequences)

SEQ ID NO: 1 - protein, 92 aa, Homo sapiens
  CARBOHYD (1)..(92): Sites of N-glycosylation at residues 52 and 78. Sites of potential nicking of internal peptide bonds after residues 1, 2, 3 and 42.
APDVQDCPECTLQEDPFFSQPGAPILQCMGCCFSRAYPTPLRSKKTMLVQKNVTSESTCCVAKSYNRVTVMGGFKVENHTACHCSTCYYHKS

SEQ ID NO: 2 - protein, 145 aa, Homo sapiens
  CARBOHYD (1)..(145): Sites of N-glycosylation at residues 13 and 30. Sites of O-glycosylation at residues 121, 127, 132 and 138. Sites of potential nicking of internal peptide bonds after residues 43, 44, 45, 47 and  75.
SKEPLRPRCRPINATLAVEKEGCPVCITVNTTICAGYCPTMTRVLQGVLPALPQVVCNYRDVRFESIRLPGCPRGVNPVVSYAVALSCQCALCRRSTTDCGGPKDHPLTCDDPRFQDSSSSKAPPPSLPSPSRLPGPSDTPILPQ

(19) United States
(12) Patent Application Publication
(10) Pub. No.: US 2006/0105411 A1
(43) Pub. Date: May 18, 2006

(54) Method of detecting early pregnancy at high accuracy by measuring hCG and hyperglycosylated hCG concentrations equally

(76) Inventor: Laurence A. Cole, Albuquerque, NM (US)

Correspondence Address: COLEMAN SUDOL SAPONE, P.C., 714 COLORADO AVENUE, BRIDGE PORT, CT, 06605-1601, US

(21) Appl. No.: 11/273,478

(22) Filed: Nov. 14, 2005

Related U.S. Application Data
(60) Provisional application No. 60/627,904, filed on Nov. 15, 2004.

(51) Int. Cl. G01N 33/53

(52) U.S. Cl. 435/7.93

(57) ABSTRACT

The present invention relates to methods for analyzing concentrations of hyperglycosylated hCG (H-hCG) and hCG equally in blood, serum or urine samples, preferably urine samples, from female patients to determine with an unexpectedly high measure of accuracy whether or not the patient is pregnant and to appropriately monitor hCG levels during the course of pregnancy. In preferred aspects of the present invention, the method utilizes an assay system which can be used in the home or at a point of care facility in a convenient manner with serum or urine samples, in a highly accurate predictive manner. It can also be used in a serum professional laboratory quantitative test.

### RELATED APPLICATIONS

This application claims the benefit of priority of provisional application serial number U.S. 60/627,904, filed Nov. 15, 2004, which is incorporated by reference in its entirety herein.

This application was made with the assistance of a grant from the National Institutes of Health. Consequently, the United States government retains certain rights in the invention.

### FIELD OF THE INVENTION

The present invention relates to methods for analyzing concentrations of hyperglycosylated hCG and hCG equally in whole blood, serum, plasma or urine samples, preferably urine samples (preferably, first urine of the day), from female patients to determine with an unexpectedly high measure of accuracy whether or not the patient is pregnant and to appropriately monitor hCG levels during the course of pregnancy. In preferred aspects of the present invention, the method utilizes an assay system which can be used in the home or at a point of care facility in a convenient manner with serum or urine samples, in a highly accurate predictive manner. It can also be used in a serum professional laboratory quantitative test.

### BACKGROUND OF THE INVENTION

Human chorionic gonadotropin (hCG) measurement is the basis of all pregnancy tests. hCG is produced by trophoblast cells of the placenta in pregnancy. It is also produced in gestational trophoblastic diseases (hydatidiform mole, choriocarcinoma and placental site trophoblastic tumors) and in testicular germ cell malignancies. hCG is a glycoprotein composed of 2 dissimilar subunits, an α- and β-subunit, held together by charge interactions. hCG β-subunit is composed of 92 amino acids and contains 2 N-linked oligosaccharides. hCG α-subunit is composed of 145 amino acids and contains 2 N-linked and 4 O-linked oligosaccharides. The 8 oligosaccharide side chains comprise >30% of the molecular weight of hCG, making it an exceptionally highly glycosylated glycoprotein.

Over 40 professional laboratory serum hCG tests, approximately 30 point of care serum and urine hCG tests and a similar number of home pregnancy tests are sold today for detecting hCG and for establishing the onset of pregnancy (1) and for monitoring the course of hCG during pregnancy. Whether intended for professional laboratory or home use, today all pregnancy tests work on the multi-antibody immunometric assay principal: commonly one and occasionally two antibodies (mono- or polyclonal) binds and immobilizes hCG and a second antibody, the tracer antibody, raised to a distant (different) epitope and labeled with an enzyme, dye or chemiluminescence agent, marks the presence of hCG or to quantitates hCG (1).

hCG is a heterogeneous molecule. Cleaved or nicked forms of hCG, free subunits of hCG, and fragments of hCG are all detectable in serum and urine samples during pregnancy (1). Variable detection or lack of detection of cleaved molecules, free subunits and fragments is a major cause of inter-assay variation in hCG results (1,2).

It has long been recognized that the hCG molecule, particularly the β-subunit of hCG, produced in choriocarcinoma (trophoblastic cancer) and testicular germ cell cancer migrates slower than hCG—subunit standards on electrophoresis gels and elutes earlier than hCG—subunit standards from gel filtration columns (3,4). This was assumed to be due to the presence of large oligosaccharides on hCG 13-subunit. Further studies with lectins have indicated the presence of irregular oligosaccharides on choriocarcinoma hCG (6,7). In 1987, we demonstrated a major difference between the 4 O-linked oligosaccharides on hCG in choriocarcinoma and normal midtrimester pregnancy hCG. The hCG from 10 normal 8-10 week gestation pregnancies primarily contained a mixture of tri- and tetrasaccharides, with 13% hexasaccharide (range 0 to 14%). In contrast, choriocarcinoma hCG preparations contained over 50% of the hexasaccharide structure (8,9). This observation was confirmed one year later by Amano et al (10).

In 1997, we showed that the difference in O-linked oligosaccharides is the principal variation between choriocarcinoma and pregnancy hCG. While normal pregnancy urine hCG contained 12.3 to 19% (mean=15.6%) hexasaccharide structures, choriocarcinoma urine hCG contained 48 to 100% (mean=74.2%) hexasaccharide structures (11). A smaller change was observed in α-subunit and β-subunit N-linked oligosaccharides (from an average of 6.8% and 14% triantennary structures in pregnancy to 9.8% and 51% triantennary structures in choriocarcinoma, on α- and β-subunit respectively (11)). We call the hCG initially identified in choriocarcinoma hyperglycosylated hCG (12). Using the choriocarcinoma preparation with 100% hexasaccharide type O-linked oligosaccharides (C5 hCG), we generated a hyperglycosylated hCG-specific antibody (antibody B152) in collaboration with Birken and colleagues (13), and established an immunoassay (12-14). In 1998 O'Connor et al. used the B152-based assay to show that hyperglycosylated hCG is also the principal form of hCG made during early pregnancy, in the weeks following implantation (14). This finding has been confirmed by these and other investigators (12, 15-17, 23).

Root trophoblast cells, or cytotrophoblasts, are invasive cells and the principal cells in choriocarcinoma tumors and in blastocysts at the time of implantation (12, 16). While cytotrophoblast produce hyperglycosylated hCG, differentiated syncytiotrophoblast cells produce regular hCG (1, 16). All of the total hCG immunoreactivity in the conditioned medium of JAR, JEG-3 and BeWo choriocarcinoma cell lines can be accounted for by hyperglycosylated hCG and hCG free 13-subunit immunoreactivity. Western blot studies with JAR, JEG-3 and BeWo hCG with enzyme-labeled Arachis hyogaea and Datura stramonium lectins clearly indicate that JEG-3 and JAR hCG were similar in sialic acid content and carbohydrate structure to the C5 hCG initially purified from choriocarcinoma patient urine (11) (Khanlian S A, Cole L A, and Butler S A, unpublished data).

A consistent standard was needed for the hyperglycosylated hCG assay using B152 antibody (marketed by Nichols Institute Diagnostics, Advantage Invasive Trophoblast Antigen (ITA) test). Culture fluid from JEG-3 cell line was selected for this purpose because hyperglycosylated hCG accounted for 100% of hCG immunoreactivity at 2 time points, reflecting sub-confluent and confluent culture densities and showing consistency with culture time. Large quantities of culture fluid were produced, and hyperglycosylated hCG was isolated. This is the only standard currently available for hyperglycosylated hCG, and can be readily isolated from JEG-3 choriocarcinoma cell line culture fluids.

hCG's primary function in pregnancy is to maintain progesterone production by corpus luteal cells (1), however, hyperglycosylated hCG is a poor stimulator of progesterone production, and clearly has an independent function. The hCG produced by JAR and JEG-3 lines of choriocarcinoma cells is hyperglycosylated hCG. Studies by Lei et al. (18) and by our laboratory (Cole L A and Dai D et al., paper submitted to Clin Cancer Res) show that JAR and JEG-3 cells are invasive in Matrigel membranes, and are tumorigenic in nude mice. Lei et al. (18) treated JAR cells with hCG β-subunit antisense cDNA and this blocked hCG (100% hyperglycosylated hCG) production. The cells which lost the ability to produce hyperglycosylated hCG, no longer were tumorigenic. In the experiments by Cole L A and Dai D, JEG-3 choriocarcinoma cells were transplanted into nude mice. Tumor rapidly grew. Tumor growth was blocked by giving mice injections of antibody against hyperglycosylated hCG. Clearly, hyperglycosylated hCG, produced by invasive trophoblast cells, has a role separate from that of regular hCG in trophoblast cell growth and invasion, whether at implantation or in choriocarcinoma.

Just as hyperglycosylated hCG is the principal form of hCG produced in invasive choriocarcinoma (primarily invasive cytotrophoblast cells), it also is the principal molecule produced in very early pregnancy, at the time of and following implantation of pregnancy (primarily invasive cytotrophoblast cells). Once implantation is complete, the invasive cytotrophoblast cells rapidly differentiate to non invasive syncytotrophoblast cells and produce regular hCG instead of hyperglycosylated hCG. For instance, hyperglycosylated hCG accounts for 97% of the total hCG immunoreactivity in serum and urine tests at the time of implantation (3^{rd} week since start of last menstrual period), 72% in the weeks that follows (4^{th} week), 50% in the following week (5^{th} week) and 25% in the following week (6^{th} week), it then very rapidly declines, to become a minor component of total hCG (<5%) through the bulk of pregnancy—see Table 1 (11, 13, 14, 23). So a clear transfer occurs between hyperglycosylated hCG and regular hCG as pregnancy progresses. Initial pregnancy testing is often performed in the 4th, 5th and 6th weeks of pregnancy, and occasionally at later times. Further, pregnancy tests are performed to follow the pregnancy. Clearly, in order to properly detect pregnancy and appropriately manage or monitor a pregnancy all tests must measure both regular hCG and hyperglycosylated hCG, ideally on an equal detection basis. A recent survey of professional laboratory quantitative serum tests, and of point of care and home pregnancy tests shown that the majority of tests optimally detect regular hCG and poorly detect hyperglycosylated hCG. A few test over recognize hyperglycosylated hCG (23). Clearly tests need to be redesigned and recalibrated or use alternative antibodies to detect the to detect regular hCG and hyperglycosylated hCG equally.

The present invention relates to a method for measuring equally both hyperglycosylated hCG concentration and hCG in early pregnancy urine samples, as compared to the sensitivities of prior art hCG tests, both professional laboratory and home pregnancy tests, to detect both hyperglycosylated hCG and hCG equally, and the importance of the equal measurement of hyperglycosylated hCG and hCG detection in all forms of pregnancy testing in order to promote extremely high accuracy in pregnancy detection. While in preliminary studies we examined home pregnancy test products in singlicate (17) and in sextuplicate (23), the present invention is an outgrowth of that study.

### OBJECTS OF THE INVENTION

It is an object of the invention to provide a method for determining pregnancy in a female patient with high accuracy regardless of the timing (whether early, middle of first trimester or anytime up to term) for testing for pregnancy, and for consistently monitoring pregnancy with hCG tests.

It is an additional object of the invention to provide a highly accurate pregnancy test which can be used at home (home pregnancy test) or at point of care facility, or quantitatively in a professional laboratory with a degree of ease.

It is still another object of the invention to provide a highly accurate pregnancy test which utilizes a single capture antibody which binds to both hyperglycosylated hCG and hCG equally in order to produce high accuracy over an extended period of pregnancy.

It is yet another object of the invention to calibrate the concentration and incubation times of capture and tracer antibodies in a pregnancy test so that they bind to both hCG and hyperglycosylated hCG equally in order to maximize the accuracy of a pregnancy test.

One or more of these and/or other objects of the invention may be readily gleaned from a careful review of the description of the invention which follows.

### BRIEF DESCRIPTION OF THE FIGURES

FIG. 1 shows the primary structure of the α- and β-subunits of hCG with carbohydrate attachment sites. See, Morgan, et al., J. Biol. Chem., 250, 5247-5258 (1975). The numbers are in amino acid sequence order. N indicates asparagine residues with N-linked oligosaccharides, and O indicates serine residues with O-linked glycans. Arrows (↑) denote sites of potential amino-terminal heterogeneity and nicking of internal peptide bonds. Molecular weight for α-subunit calculated based on an intact primary sequence, five disulfide bonds, one sialylated monoantennary and one sialylated biantennary.

### SUMMARY OF THE INVENTION

The present invention provides methods for detecting pregnancy in a woman with extremely high accuracy from as early as the 3^{rd} week of pregnancy through the 11^{th} week of pregnancy (i.e., after the woman's last menstrual cycle) or later through to term. In particular, the methods comprise screening biological samples for biological markers associated with pregnancy, namely hyperglycosylated hCG (H-hCG) and hCG equally using a single capture antibody and screening so that both hyperglycosylated hCG and hCG are equally measured. In one aspect of the invention, using a combination of a single capture antibody which binds both hyperglycosylated hCG and hCG, preferably both intact hyperglycosylated hCG and hCG, as well as the β-subunit of hyperglycosylated hCG and hCG, but which is calibrated (by varying the concentration of antibody and the incubation time of the assay) to bind equally hyperglycosylated hCG and hCG (get an equal measurement) in one assay in combination with one or more detection antibodies dramatically improves the sensitivity of the assay for the biological markers of pregnancy for an unexpectedly long period. The accuracy of pregnancy tests according to the present invention is unexpectedly (at least about 95%, preferably at least about 98%, preferably at least about 99% and preferably about 100% accuracy) high over a broad time frame—from the 4^{th} week (ie, after the patient's last menstrual cycle) through the 11^{th} week of pregnancy and beyond.

In one embodiment of the invention, a method for detecting pregnancy in a woman comprises the steps of: contacting a biological sample of the woman with a single capture antibody which has been calibrated to bind both hyperglycosylated hCG (i.e., H-hCG) and hCG or intact H-hCG and hCG, as well as β-subunits of H-hCG and hCG, equally, and at least one detection antibody which has been calibrated to bind both H-hCG and hCG, or alternatively intact hyperglycosylated hCG and hCG as well as β-subunits of hyperglycosylated hCG and hCG equally such that the concentration of hyperglycosylated hCG and hCG and, where applicable, additionally the β-subunit of hyperglycosylated hCG and hCG in the sample may be measured equally, in an assay, preferably a calorimetric or chemiluminescent assay. In this aspect of this invention, the assay comprises no more than one capture antibody which specifically binds hyperglycosylated hCG and hCG or both intact hyperglycosylated hCG and hCG as well as the β-subunit of hyperglycosylated hCG and hCG respectively and at least one or more detection antibody, each of which antibodies have been calibrated to bind the antigens in a manner such that the concentration of hyperglycosylated hCG and hCG or its β-subunits in the sample may be measured (detected) equally. In preferred aspects of the present invention a label coupled to the capture antibody and/or the detection antibody (preferably the detection antibody alone) produces a detectable signal. The assay is preferably a calorimetric or chemiluminescent assay. The assay is more preferably a point of care assay, including an over the counter (OTC) home pregnancy colorimetric assay.

### DETAILED DESCRIPTION OF THE INVENTION

The following terms shall be used throughout the specification to describe the present invention.

The term “patient” is used throughout the specification to describe an animal, preferably a mammal, more preferably a human, to whom an analysis of pregnancy according to the present invention is provided. For a particular analysis to be provided, the term patient refers to that specific animal, most often a human female patient.

The term “sample” shall mean a whole blood, serum, plasma or urine sample, preferably a serum or urine sample (if urine, most preferably the first urine sample of the day), taken from a patient and used to determine whether said patient is pregnant.

The term “effective amount” is used throughout the specification to describe an amount of an antibody or other component, including a sample taken from a patient, which is used in an assay according to the present invention to effect an intended result when used in the method of the present invention.

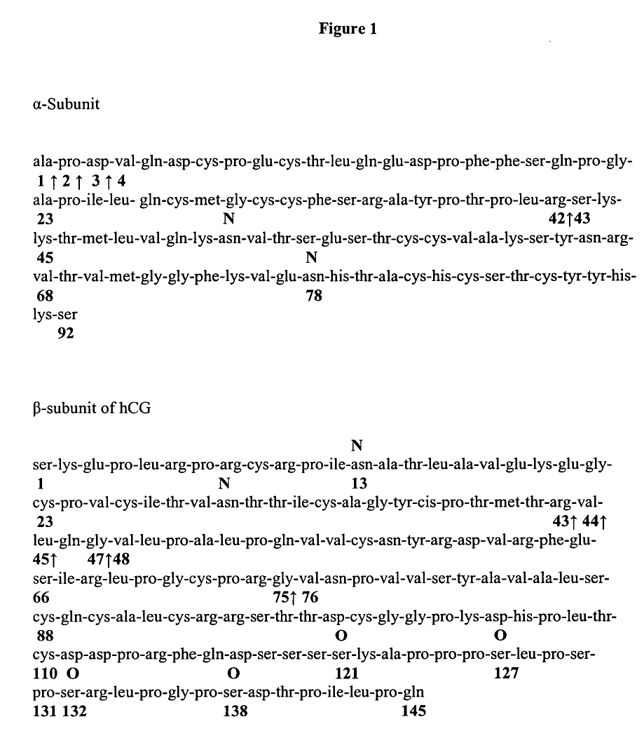

The terms “hyperglycosylated hCG”, “H-hCG”, “invasive trophoblast antigen” and “ITA” are used interchangeably throughout the specification to describe a glycoprotein hormone secreted by trophoblast cells of the placenta of pregnant women and by cancer cells. Hyperglycosylated hCG is also called hyperglycosylated ITA. H-hCG is similar to C5 hCG, which is a nicked h-hCG obtained from a choriocarcinoma patient. In particular, H-hCG encompasses molecules that exhibit similar biological activities or expression patterns to H-hCG and that exhibit aberrant carbohydrate levels as compared to normally glycosylated hCG including, nicked hCG, β-subunits of hyperglocosylated hCG (“β-ITA”), or any combination thereof. Examples of hyperglycosylated hCG isoforms include isoforms that comprise 57% triantennary N-linked oligosaccharides and 68% hexasaccharide-type O-linked oligosaccharides. Another hyperglycosylated hCG isoform may comprise 48% triantennary N-linked oligosaccharides and 100% hexasaccharide-type O-linked oligosaccharides or alternatively, for example during pregnancy, a relatively small proportion of more complex triantennary N-linked oligosaccharides (0-30%) and larger hexasaccharide-type O-linked sugar units (0-20%) are also found. Representative chemical structures of hCG, hyperglycosylated hCG, β-hCG and β-hyperglycosylated hCG are set forth in attached FIG. 1.

The term “calibrated” is used to describe the process by which an antibody which is used in assays according to the present invention is screened for concentration and reaction time such that the antibody used in the assay will actually react with both hCG and H-hCG equally (i.e. wherein the immunoreactivity with one antigen is at least about 90%, preferably at least about 95%, preferably at least about 98%, preferably at least about 99% and preferably about 100% of the reactivity of the other antigen(s) to be measured. The assays are then modified to accommodate the calibrated antibody so that the assay may equally measure hCG and H-hCG or hCG, H-hCG and the β-subunit of these antigens in a given sample. The present invention utilizes assays for both hCG and H-hCG (which may also measure the β-subunit of these antigens) which comprise a single capture monoclonal assay which measures both hCG and H-hCG (and optionally, the β-subunits of these antigens) essentially equally utilizing a single capture monoclonal antibody which has been calibrated to bind to hCG and H-hCG (and optionally the β-subunits of hCG and H-hCG) equally and at least one type of detection antibody which also has been calibrated to react equally with both hCG and H-hCG (and optionally the β-subunits of hCG and H-hCG). It is noted here that when more than one type of antibody is used for the detection antibody, the antibodies are calibrated such that each of the detection antibodies or alternatively, the sum of the detection antibodies produces a substantially equal binding to both hCG and H-hCG. The antibodies according to the present invention are calibrated using standards of hCG and H-hCG or hCG and H-hCG and their β-subunits, and then the concentration of antibody and/or the incubation time of the antibody is varied in order to calibrate the antibody to equal reactivity with both hCG and H-hCG or hCG and H-hCG and their β-subunits. By calibrating the capture antibody and the one or more detection antibodies to be equally reactive with hCG and H-hCG, the assays may be maximally accurate (at least about 95% accurate, approaching 100% accuracy) from about the 4^{th} week of pregnancy to beyond the 11^{th} week of pregnancy (measured from the patient's last menstrual period).

When calibrating the present invention, the following 3 criterion are used to choose the best antibody concentrations and incubation time—

1. Provide a background measurement, or result with 0 ng/ml standard.

2. Provide a range of values, slope of spectrometric result (from enzyme-labeled, colorimetric or chemiluminescence, reaction, etc.) with change in concentration, the steeper the result the more accurate the test.

3. The assay must provide similar or preferably, identical spectrometric results with different concentration of hCG and with hyperglycosylated hCG. In the event that this cannot be achieved, while still satisfying criterion 1 and 2, then the chosen antibodies are inappropriate and a change of antibodies is indicated.

Without being limited by way of theory, it is believed that many of the monoclonal antibodies which are raised to hCG will also be reactive to H-hCG and many of the monoclonal antibodies which are raised to β-hCG will be reactive to the β-subunit of H-hCG, and in many cases also to intact hCG and H-hCG. The relative immunoreactivity of an antibody (polyclonal or monoclonal) to hCG and H-hCG will be a function of the site of the epitope and the extent of glycosylation at or near that epitope which might otherwise inhibit to some extent the binding of antibody to the antigen. By calibrating the antibody to overcome the differential immunoreactivity of the antibody to intact hCG or H-hCG or the β-subunit of these antigens by changing the concentration of the antibody and/or the incubation time of the antibody with the antigen (generally, by raising the concentration of the antibody above a threshold level and increasing the incubation time beyond a threshold level), one can in fact, produce an assay which will bind substantially equally (and maximally) to hCG and H-hCG. In the present invention, the capture antibodies which are chosen for use in the pregnancy tests have an immunoreactivity with the antigens to be measured which is substantially equal, i.e., at least about 90% (preferably, at least about 95%, preferably at least about 98%, preferably at least about 99% and preferably about 100%) of the immunoreactivity of the antibody with the other antigens to be measured in the assay. In the present invention, in order to provide for maximum accuracy of the pregnancy test, it is preferred that the antigens to be measured include intact hCG, intact H-hCG, the β-subunit of hCG and H-hCG and that capture antibody used in the assay should be calibrated to react substantially equally with these four antigens.

The term “accuracy” shall refer to results of assays employing the methods of the present invention. The present methods are accurate to a level of at least about 95%, which means that the tests will correctly predict pregnancy at least 95% of the time with virtually no false positives. Preferably, the accuracy of the pregnancy test methods according to the present invention is at least about 98%, preferably at least about 99% and in many instances will approach 100%.

The term “antibody” shall mean an antibody, or an antigen-binding portion thereof, that binds to H-hCG or hCG; preferably both H-hCG and hCG (especially for the capture antibody according to the present invention, but preferably both the capture antibody and the detection antibody), and in certain embodiments both intact H-hCG and hCG, as well as the β-subunit of H-hCG and hCG. The term “antibody” refers to a polypeptide substantially encoded by an immunoglobulin gene or immunoglobulin genes, or fragments thereof, which specifically recognize and bind H-hCG or hCG, or both H-hCG and hCG. The recognized immunoglobulin genes include the kappa, lambda, alpha, gamma, delta, epsilon, and mu constant region genes, as well as the immunoglobulin variable region genes. Antibodies include fragments, such as Fab′, F(ab)₂, Fabc, and Fv fragments. The term “antibody,” as used herein, also includes antibody fragments either produced by the modification of whole antibodies or those synthesized de novo using recombinant DNA methodologies, and further includes “humanized” antibodies made by now conventional techniques.

The antibodies may be used as capture antibodies or as detection antibodies in pregnancy assays according to the present invention. The antibodies may be polyclonal or monoclonal, but are preferably monoclonal because of the ability of the monoclonal antibody to be more readily calibrated and standardized for binding equally to H-hCG and hCG, or to intact H-hCG and hCG as well as the β-subunit of H-hCG and hCG and used as the capture antibody in the present invention. The monoclonal antibody may be human or non-human and may be murine, rat or rabbit derived. Methods for making polyclonal and monoclonal antibodies are well known to the art. Monoclonal antibodies can be prepared, for example, using hybridoma techniques, recombinant, and phage display technologies, or a combination thereof. See, for example, Golub et al., U.S. Patent Application Publication No. 2003/0134300, published Jul. 17, 2003, for a detailed description of the preparation and use of antibodies as diagnostic agents. Antibodies to hCG and hCG isoforms, such as H-hCG (ITA), can be generated by standard means as described, for example, in “Antibodies: A Laboratory Manual” by Harlow and Lane (Cold Spring Harbor Press, 1988), relevant portions of which are hereby incorporated by reference.

Preferably, the antibody is a monoclonal antibody to provide the desired specificity and, in the case of the capture antibody useful in the assay aspect of the present invention, the monoclonal should be reactive with both H-hCG and hCG, or to intact H-hCG and hCG as well as the β-subunit of H-hCG and hCG, substantially equally and preferably as close to equally, as is possible. The monoclonal antibody preferably useful as a capture antibody in the present invention is one which is reactive to epitopes which are common to both H-hCG and hCG. These are the preferred antibodies for use according to the present invention. Monoclonal antibodies which are useful in this aspect of the present invention may be reactive with the α-subunit or β-subunit of both H-hCG and hCG, including either the β₁ core of the β-subunit and/or the β₂ core of the β-subunit of both H-hCG and hCG. These monoclonals are respectively anti-α-subunit, anti-β₁ core and/or anti-β₂ core, anti-C-terminal (which may be raised from synthetic peptides, which are identical to, for example, amino acids 79-92 of the α-subunit or amino acids 131-145 of the β-subunit of hCG and H-hCG—without the glycosylation—see FIG. 1). These antibodies, with the exception of the anti-C-terminal antibodies, are preferably raised against the intact molecules of H-hCG and hCG in order that the resulting antibodies will be reactive with intact molecules of H-hCG and hCG in the assay.

An antibody “specifically binds to” or “is immunoreactive with” a protein when the antibody functions in a binding reaction with the protein. In order for the antibody to bind to a protein, the protein should contact the antibody. Accordingly, contacting a sample suspected of containing an antigen of interest with an antibody to the antigen will permit the antibody to specifically bind the antigen. The binding of the antibody to the protein permits determination of the presence of the protein in a sample in the presence of a heterogeneous population of proteins and other agents. Thus, under designated immunoassay conditions, the specified antibodies bind preferentially to a particular protein and do not significantly bind to other proteins present in the sample. Specific binding to a protein under such conditions requires an antibody that is selected for specificity for a particular protein. Several methods for determining whether or not a peptide is immunoreactive with an antibody are known in the art.

As used herein, a “capture antibody” is defined as an antibody, preferably a monoclonal antibody, attached to a substrate, such as a solid substrate. The capture antibody is selected to specifically bind a particular, distinct epitope of both H-hCG and hCG. By utilizing a single capture antibody and measuring both H-hCG and hCG using that single capture antibody, a very accurate pregnancy test may be formulated which can be used most preferably in a home pregnancy test.

As disclosed herein, a single capture antibody, which antibody is immunoreactive with both hCG and H-hCG is used in the present assays and in preferred embodiments is attached to a solid substrate, which may be a polymeric resin or other solid support. Any number of monoclonal antibodies which bind both hCG and H-hCG may be used. Exemplary antibodies which may be used in the assay according to the present invention as the capture antibody, include, for example, the antibody designated H222 (available from Johnson & Johnson) and B-207 (ATCC Accession Number PTA 1626), which are immunoreactive with the β₁ core of both hCG and H-hCG; the antibody designated 2119-12, which is available from Unipath, Inc., London UK and is immunoreactive with the α-subunit of both hCG and H-hCG, other anti-β₂ core antibodies, such as that available from DPC, Inc., and the monoclonal antibodies reactive with the C-terminal peptide of the α-subunit or the β-subunit of both hCG and H-hCG, as otherwise described herein.

Another capture antibody which can be used to practice the methods of the invention is the publicly available monoclonal antibody clone 820 available from Biodesign International, Saco, Me. (Catalog Number E45550M). Clone 820 is a monoclonal antibody to hCG. Clone 820 specifically binds to intact hCG (cross reactivity is 100%). The cross reactivity with beta-hCG is less than 1.0%, with alpha-hCG is less than 1.0%, with luteinizing hormone is less than 0.1%, with thyroid stimulating hormone is less than 0.1%, and with follicle stimulating hormone is less than 1.0%, but also binds with H-hCG (see U.S. Pat. No. 6,627,457, which is incorporated by reference herein). Clone 820 was produced in mouse, and is an IgG1 isotype. The hybridoma was prepared by fusing myeloma cells with spleen cells from Balb/c mice. Purified Clone 820 is stored in liquid format at a concentration of 5.64 mg/mL in 0.015 M potassium phosphate buffer, 0.15 M NaCl, at a pH of 7.2. The preservative is 0.1% sodium azide.

Another capture antibody which may be used to practice the methods of the present invention is the publicly available monoclonal antibody clone 827 available from Biodesign International, Saco, Me. (Catalog Number E45575M). Clone 827 is a monoclonal antibody to the beta subunit of hCG. Clone 827 specifically binds to beta-hCG (cross reactivity is 100%). The cross reactivity with intact hCG is 0.5%, with alpha-hCG is less than 0.1%, with luteinizing hormone is less than 0.1%, with thyroid stimulating hormone is less than 0.1%, and with follicle stimulating hormone is less than 0.1%. However, as described in Example 3, infra, Clone 827 may also specifically bind H-hCG, because the H-hCG standards were reactive with the Clone 827. Clone 827 was produced in mouse, and is an IgG1 isotype. The hybridoma was prepared by fusing myeloma cells with spleen cells from Balb/c mice. Purified Clone 827 is stored in liquid format at a concentration of 4.44 mg/mL in 0.015 M potassium phosphate buffer, 0.15 M NaCl, at a pH of 7.2. The preservative is 0.1% sodium azide.

As used herein, a “detection antibody” is defined as an antibody, preferably a monoclonal antibody, that binds an antigen at a binding site or epitope distinct from that of the capture antibody. As is understood in the art, depending on the amount of cross-reactivity that is desired for related antigens, the specificity of the detection antibody may vary. For example, and as discussed herein, for combination assays where two or more antigens are assayed, it may be desirable to use two capture antibodies that specifically bind each antigen, and one detection antibody that will bind an epitope similar or identical on both antigen molecules.

In certain embodiments of the invention, the detection antibody is a monoclonal antibody that recognizes hCG and/or H-hCG and comprises a label for detecting the monoclonal bound to hCG and/or H-hCG in the assay system. Alternatively, the detection antibody may comprise two or more antibodies, within this context, preferably no more than two antibodies, which may be calibrated so that the antibodies bind both hCG and H-hCG equally. One example is a monoclonal antibody designated B207, described above, which is reactive with the B₁ core of hCG and H-hCG. Monoclonal antibody B207 was generated to the beta subunit of hCG, but is cross reactive with the beta subunit of H-hCG. The hybridoma producing the B207 monoclonal antibody was deposited with the American Type Culture Collection (ATCC), 12301 Parklawn Drive, Rockville, Md. 20852, U.S.A. under the provisions of the Budapest Treaty for the International Recognition of the Deposit of Microorganism for the Purposes of Patent Procedure. The hybridoma was accorded ATCC Accession Number PTA 1626. The B207 mAb was developed and described in Krichevsky et al., (1994) The Development of a Panel of Monoclonal Antibodies to Human Luteinizing Hormone and its Application to Immunological Mapping and Two-Site Assays, Endocrine, 2:511-520; WO 99/41584, Methods for Predicting Pregnancy Outcome in a Subject by hCG Assay; and WO 00/70094, Methods for Predicting Pregnancy Outcome in a Subject by hCG Assay; O'Connor et al., (1998) Differential Urinary Gonadotrophin Profiles in Early Pregnancy and Early Pregnancy Loss, Prenatal Diagnosis, 18:1232-1240.

Other antibodies which may be useful as detection antibodies include, for example, antibodies raised to anti-C-terminal (which may be raised from synthetic peptides, which are identical to, for example, amino acids 79-92 of the α-subunit or amino acids 131-145 of the β-subunit of hCG and H-hCG—without the glycosylation—see FIG. 1). Numerous additional antibodies which bind both hCG and H-hCG and are well known in the art may be used provided that the antibodies may be calibrated in concentration and/or incubation time to bind to hCG and H-hCG equally.

In preferred aspects of the present invention, the detection antibody is coupled to a label, as described herein. The concentration of detection antibody used in practicing the methods of the invention is predetermined and optimized by conducting experiments to determine amounts of detection antibodies that are needed to provide a detectable signal and then further calibrating to make certain that the detection antibody will bind to hCG and H-hCG equally or to intact H-hCG and hCG as well as the β-subunit of H-hCG and hCG equally. Otherwise, either hCG or H-hCG or the respective β-subunit of each antigen will be more fully bound to the detection antibody and the antigen more fully bound will be more readily detected in the assay—a condition which may impair accuracy of the assay.

It will be understood by persons skilled in the art that a sufficient concentration of detection antibody is provided and the antibody is further calibrated for incubation times to ensure binding of the detection antibody to all, or essentially all, of the test antigen molecules, in this case both hCG and H-hCG. In other words, it is preferable to use as much detection antibody as possible without increasing non-specific binding of the detection antibody in the assay to improve the signal-to-noise ratio of the device of the invention.

Preferably, a single detection antibody is used in assays to minimize non-specific binding and erroneous detection of non-antigens in the assays. However, in certain embodiments of the invention, detection antibodies may be monoclonal antibodies that specifically bind two different epitopes of hCG and H-hCG or intact H-hCG and hCG as well as the β-subunit of H-hCG and hCG. For example, the two detection antibodies may bind an epitope on the beta subunit of intact hCG and intact H-hCG and an epitope on the alpha subunit of intact hCG and intact H-hCG. Alternatively, the two detection antibodies may bind two different epitopes on the alpha subunit or two different epitopes on the beta subunit of intact hCG and intact H-hCG, which antibodies may also bind to the same epitopes on the free β-subunit of hCG or H-hCG. Examples of detection antibodies which may be used include monoclonal antibodies as described hereinabove. Other antibodies may be produced and screened using conventional immunological techniques. The detection antibodies may be monoclonal antibodies that bind the antigen at an epitope that does not interfere with the binding of the capture antibodies to the antigen. The detection antibodies can be relatively less specific than the capture antibodies, although they must ultimately bind equally to hCG and H-hCG or to intact H-hCG and hCG as well as the β-subunit of H-hCG and hCG, those antigens are measured in the assay, after calibration for concentration and incubation time. In one embodiment of the invention, the detection antibody is designated B207, as described herein.

The detection antibody is coupled to a label, as described herein. The concentration of detection antibody used in practicing the methods of the invention is predetermined and optimized by conducting experiments to determine amounts and incubation times of detection antibodies that are needed to provide a detectable signal. It will be understood by persons skilled in the art that a sufficient concentration of detection antibody is provided to ensure binding of the detection antibody to all, or essentially all, of the test antigen molecules. In other words, it is preferable to use as much detection antibody as possible without increasing non-specific binding of the detection antibody in the assay to improve the signal-to-noise ratio of the device of the invention.

In the present invention, preferred capture antibodies are monoclonal antibodies that specifically bind a single epitope on both hCG and H-hCG or on intact H-hCG and hCG as well as the β-subunit of H-hCG and hCG. For example, the capture antibody may bind an epitope on the alpha- or beta subunit of H-hCG and hCG. In the present invention, a single capture antibody is used preferably because it leads to maximum accuracy (at least about 95% and preferably, about 99-100%) without increasing the cost associated with the use of more than one capture antibody. In contrast, the detection antibodies may be monoclonal antibodies that bind the antigen at an epitope that does not interfere with the binding of the capture antibody to the antigen. The detection antibodies can be relatively less specific than the capture antibodies, although it is noted that the detection antibody must bind substantially all of the hCG and H-hCG or intact H-hCG and hCG as well as the β-subunit of H-hCG and hCG molecules in the sample if those are measured.

A “label” is a composition detectable by spectroscopic, photochemical, biochemical, immunochemical, or chemical means. In other words, a label produces a detectable signal in practicing the methods of the invention. For example, useful labels include fluorescent dyes, chemiluminescent compounds, radioisotopes, electron-dense reagents, enzymes, colored particles, biotin, or dioxigenin, among others. A label often generates a measurable signal, such as radioactivity, fluorescent light, color, or enzyme activity, which can be used to quantitate the amount of bound label.

Examples of chemiluminescent compounds include luciferin, a luminol derivative, pyrogallol, isoluminol, aequorin, cyclic arylhydrazides, dioxetanes, rhodium chelates (electrochemiluminescent), oxalate esters, thermochemiluminescent labels, acridinium and the like. These labels may be attached to a protein, for example an anti-hCG or anti-H-hCG antibody, using techniques well known in the art. (See U.S. Pat. No. 5,284,952, the disclosure of which is incorporated in its entirety herein by reference.) In one embodiment, a detection antibody, such as B207, may be labeled with an acridinium ester by employing the methods found in U.S. Pat. Nos. 5,284,952, 5,110,932, and 5,338,847, the disclosures of which are incorporated in their entirety herein by reference. In other embodiments the detection antibody is labeled with a gold dye or other dye which can function in a colorimetric assay. The preferred assay for use in the present method is a calorimetric assay.

Examples of the fluorescent material to be used for labeling include fluorescein, fluorescamine, fluorescein isothiocyanate, umbelliferone, rhodamine, Texas red dyes, pthalocyanines, coumarin, squaraine, anthracene, erythrosine, europium chelates and the like.

Examples of radioactive isotopes to be used for labeling include ¹⁴C, ³H, ³²P, ¹⁸F or ¹²⁵I.

Exemplary enzymes which have been developed and can be used in assays of the invention are those described in U.S. Pat. Nos. 3,654,090; 3,791,932; 3,839,153; 3,850,752; 3,817,837; 3,879,262; Journal of Immunological Methods 1: 247 (1972); and the Journal of Immunology 109:129 (1972), the disclosures of which are incorporated in their entirety herein by reference. Other examples of enzymes include, but are not limited to, alkaline phosphatase, beta galactosidase, horseradish peroxidase, gluconidase, phosphatase, peptidase, alkaline phosphatase and the like. Co-enzymes useful in this invention include molecules and/or proteins which facilitate an enzyme to catalyze a reactant to produce a detectable product, for example light. A co-enzyme may include, without limitation, FAD and NAD.

Examples of colored particles include colloidal gold, or blue latex.

Other labels may include a non-active precursor of a spectrophotometrically-active substance (British Pat. No. 1,392,403 and French Pat. No. 2,201,299, which patents correspond to U.S. Pat. No. 3,880,934) and electron spin resonance moieties (U.S. Pat. No. 3,850,578).

As described herein, certain parameters of the assays used to practice the methods of the invention are determined prior to practicing the methods. For example, the components of the solutions and their concentrations (e.g., the concentrations of capture and detection antibodies); the experimental conditions of the assays, such as buffer solution, pH, ionic strength, temperature, incubation times, solid phase support; the coupling chemistry between the support and the various antibodies, and the coupling chemistry between the detection antibody and the label, are preferably predetermined by conducting conventional experiments to optimize the methods of the invention.

The present invention is, in part, based upon the discovery that pregnancy may be detected with increased sensitivity and accuracy by measuring a woman's level of both hCG and H-hCG with a single capture antibody in combination with one or more detection antibodies.

The methods for detecting pregnancy disclosed herein comprise contacting a biological sample of a woman with antibodies that bind to hCG or H-hCG, alone or in combination with other antigens or biological markers (pregnancy markers), and comparing the amount of measured antigens (e.g., hCG and H— hCG) to a standard that has been determined to reflect the likelihood of a woman being pregnant. The present method is useful for detecting pregnancy with near 100% accuracy (at least about 99-100% accuracy) from the 3^{rd} week of pregnancy until at least the 7^{th} or 8^{th} week of pregnancy or more, although the present invention may be useful for determining pregnancy through the 9^{th}-12^{th} week of pregnancy.

In the present application, the measurement of intact H-hCG and hCG or both intact H-hCG and hCG as well as the β-subunits of hCG and H-hCG is performed to determine that a woman is pregnant. In the present invention, this amount at the earliest stage that pregnancy can be predicted (about 3-4 weeks after the patient's last menstrual cycle) is at least about 2 miu/ml for professional tests and about 6 mIU/ml for point of care tests, above which the test will indicate a positive result for pregnancy. (IU means International Units; 11 mIU/ml is 1 ng/mL; 2 mIU/mL corresponds to approximately 0.18 ng/mL of hCG; 6 mIU/ml corresponds to approximately 0.55 ng/mL). Thus, the above concentrations of hCG and H-hCG is typically an indication that the woman being tested is pregnant. It is believed that H-levels increase before the increase in hCG levels, so the above cutoff points for determining pregnancy, effective at the early date of about 4 weeks after the patient's last menstrual cycle, are appropriate for determining pregnancy at a later time in pregnancy, when hCG levels are significantly higher. Thus, measuring the concentration of H-hCG and hCG in a biological sample provides a marker to detect pregnancy before hCG levels increase as well as when hCG levels increase.

Biological samples useful for practicing the methods of the invention include, but are not limited to, whole blood, serum, urine, plasma, and amniotic fluid. In addition, the samples may include tissue samples, such as, for example, tissue from the placenta, vagina, or uterus of a pregnant woman. In one embodiment of the invention, the biological sample is urine. In professional pregnancy tests, the use of serum and plasma may be preferred in order to provide greater accuracy in measurement. Urine provides an ease or convenience, but because of factors which include dilution, may be less consistent than serum or plasma samples. Urine samples are preferred for home pregnancy or point of care pregnancy tests.

Samples may be obtained from pregnant women by any conventional method known to those skilled in the art. For example, serum samples may be obtained by withdrawing a volume of blood from the pregnant woman using conventional intravenous techniques. Amniotic samples can be obtained by withdrawing amniotic fluid from pregnant women using a needle and syringe. Urine samples can be obtained from the pregnant woman.

In one embodiment, screening the biological sample for H-hCG and hCG may be performed by contacting the sample with antibodies that specifically bind H-hCG and hCG. In other embodiments, especially at an early stage of pregnancy, for example, in the first few weeks of pregnancy, the use of a capture antibody which binds to the β-subunit (either the β1 or the β₂ core) of hCG and H-hCG measures not only intact hCG and H-hCG, but also free β-subunit hCG and H-hCG.

In one embodiment of the invention, “sandwich” type immunoassays are utilized to measure hCG and H-hCG in a sample. The methods of the invention utilize a capture antibody that specifically binds to both hCG and H-hCG which has been calibrated according to concentration and incubation times to bind equally to both hCG and H-hCG. The capture antibody may be coupled to a solid substrate or solid phase. Examples of suitable substrates include, but are not limited to, wells of microtiter plates or cuvettes, or nitrocellulose or nylon membranes. In one embodiment of the invention, the capture antibodies are coupled to paramagnetic particles in wells of microtiter plates or cuvettes. For example, biotin-coupled capture antibodies can couple to streptavidin coated paramagnetic particles via the well known avidin-biotin binding reaction. Other methods of coupling the capture antibody to the solid phase of the assays are known to those skilled in the art. In certain embodiments of the invention, the capture antibody is designated the antibody designated H222 (available from Johnson & Johnson) and B-207 (ATCC Accession Number PTA 1626), which are immunoreactive with the β₁ core of both hCG and H-hCG are utilized. The use of these and/or other antibodies which are otherwise described herein or are well known in the art may be employed by analogy according to the teachings of WO 98/10282, Prenatal Screening for Down's Syndrome Using Hyperglycosylated Gonadotropin; WO 99/41584, Methods for Predicting Pregnancy Outcome in a Subject by hCG Assay; WO 00/70094, Methods for Predicting Pregnancy Outcome in a Subject by hCG Assay; O'Connor et al., (1998) Differential Urinary Gonadotrophin Profiles in Early Pregnancy and Early Pregnancy Loss, Prenatal Diagnosis, 18:1232-1240; Cole et al., (1999), among numerous others.

In practicing the sandwich immunoassay, hCG and H-hCG may also be exposed to a detection antibody that is coupled to a detectable label. Examples of suitable labels are described above, one example of a label is an acridinium ester. Methods of coupling labels to antibodies are well known in the art. For example, acridinium, as a “sulfonyl chloride ester” can be crosslinked to the detection antibody by the reaction of the lysly moiety of the epsilon amino group of lysine in proteins, such as antibodies, to the acridinium ester. The reaction products may then be separated by size exclusion chromatography on Sepharose beads. One detection antibody is designated B207—others are know in the art. B207 was developed to the hCG β-fragment as described in Krichevsky et al., (1994) The Development of a Panel of Monoclonal Antibodies to Human Luteinizing Hormone and its Application to Immunological Mapping and Two-Site Assays, Endocrine, 2:511-520.

In certain embodiments of the invention, the sandwich immunoassays may be chemiluminescent immunoassays, but colorimetric assays are preferred for point of care applications. The range of sensitivity of concentration of the assays disclosed herein is from at least about 1 mIU/ml (about 0.09 ng/mL), more preferably at least about 2 mIU/ml (about 0.18 ng/mL) for a professional assay to at least about 6 mIU/ml (about 0.55 ng/mL) for a point of care application, which includes a home pregnancy test. Even lower sensitivities can be determined using the methods of the present invention.

Although specific monoclonal antibodies are disclosed herein, other monoclonal antibodies that could be used as capture and detection antibodies for both hCG and H-hCG as described herein can be produced using conventional methods known in the art. See, for example, Kohler and Milstein, (1975) Nature, 256:495-97; or Sambrook et al. (2001) Molecular Cloning: A Laboratory Manual, 3rd edition, Cold Spring Harbor Laboratory Press. Briefly, animals, such as mice, are injected with an antigen, such as hCG, H-hCG, or fragments thereof, that may be coupled to a carrier protein. The animals are boosted with one or more antigen injections, and are hyperimmunized by an intravenous (IV) booster about three days before fusion. Spleen cells from the mice are isolated and are fused by standard methods to myeloma cells. Hybridomas are selected in standard hypoxanthine/aminopterin/thymine (HAT) medium, according to standard methods. Hybridomas secreting antibodies which recognize different epitopes of the antigen are identified, cultured, and subcloned using standard immunological techniques. The antibodies are then screened for the desired specificity or cross reactivity using methods known in the art.

Although one embodiment of the invention employs colorimetric or chemiluminescent sandwich inumunoassays to practice the methods of the invention, other immunoassays, such as ELISAs and RIAs may be used. The parameters and components of the assays are determined and optimized as is well known to those skilled in the art such that the assays provide measurement of hCG and H-hCG levels in the biological samples being assayed. In addition, although certain embodiments of the invention utilize antibodies as the agents capturing the hCG and H-hCG, hCG and H-hCG may be captured in the assays of the invention using other chemical agents or molecules that are not antibodies. For example, such an agent may recognize carbohydrate profiles of hCG and H-hCG, and thereby bind the hCG and H-hCG to a solid phase in a similar manner as the capture antibodies described herein.

In some embodiments of the invention, it may be desirable to automate the methods as much as practical in order to improve replicability of the results and reduce the time and costs required to conduct the assays. Automated assays used to practice the methods of the invention permit users to conduct at least about 80 tests per hour, and preferably more than about 100 test per hour.

One may also use any conventional, non-automated, assay device to practice the methods of the invention. For example, a conventional microtiter plate can be used to store the various solutions used in performing the assay. The device should permit the biological sample to be exposed to a combination of antibodies. The antibodies may recognize different epitopes of the antigen(s) being assayed. The device should also cause the bound antigen to be retained to a substrate as solutions are added and removed during the assay.

By way of example, and not by way of limitation, wells of a microtiter plate can be loaded with a solution containing streptavidin coated magnetic particles, as described herein.

A solution containing biotin coupled capture antibodies (e.g., biotin coupled mAb) is added to the well to enable the coupling of the capture antibodies to the magnetic particles. A concentration of capture antibody is empirically selected (based on expected antigen concentrations) as discussed herein, to permit binding of all, or essentially all, of the test antigen that is available in the sample. In that regard, typical antigen concentrations in biological samples are in the nanogram to low microgram range (e.g. less than 1 ng/ml-5 μg/ml) so that the capture antibody concentrations are in the low to high microgram range (e.g. 1-100 μg/ml). The sample is added to the well. If the sample contains the antigens of interest (e.g., H-hCG and hCG), the antigen will bind to the capture antibodies. The plate is exposed to a magnetic field to immobilize the magnetic particles, and the solution is removed from the well; but the antigen will not be removed because it is bound to the antibodies that are bound to the magnetic particles that are immobilized by the magnetic field. A solution containing the detection antibody coupled to a label (e.g., acridinium labeled mAb) is added to the well containing the bound antigen. As indicated elsewhere herein, the concentration of the detection antibody is preferably selected so that all, or essentially all, of the test antigen molecules (e.g., hCG and H-hCG) are bound by the detection antibody. Thus, the detection antibody can be provided at concentrations at least an order of magnitude greater than the expected concentration of the test antigen. For example, if a test antigen has an expected concentration of 2 ng/ml, the detection antibody concentration can be at least 20 ng/ml (0.02 μg/ml). After a sufficient amount of time (from about 10 minutes to about 8 or more hours, which is determined in a calibration step), determined and optimized empirically as described herein, the plate is exposed to a magnetic field, the solution is then removed, and the sample is washed. The amount of label remaining in the well is then measured (e.g., by a luminometer). The measured values can be quantitative or qualitative. Quantitative results are usually preferred. The measured values may then be compared to a standard or a threshold.

One immunoassay system which may be used in the present invention is the Nichols Advantage.RTM. immunoassay system, which is a fully automated chemiluminescent system. The system is a bench-top instrument that performs solid phase chemiluminescent immunoassays. Streptavidin-coated magnetic particles and biotinylated antibodies may be employed in the assay system. Acridinium ester is typically the chemiluminescence label for signal detection. Other assay systems may also be employed in the present method.

In practicing the methods of the invention, a control may be provided in the assay to ensure that the reactions have been successful. For example, a control could be provided with a polyclonal antibody solution for other analytes present in the biological sample. A specific example could be to detect the presence of progesterone, or metabolites thereof, in the sample. If the methods are practiced and the test results for the sample and the control are negative, or if the sample is positive and the control is negative (e.g., there is no detectable signal), it is likely to mean that the woman was either not pregnant to begin with, that an error has been made in the testing protocol, or that the test materials have been compromised in some manner. Alternatively, if a signal is detected in the sample reaction zone and in the control, it is likely that the woman is pregnant.

In point of care applications, and in home pregnancy tests, the following exemplary calorimetric assay may be used. The assay may also be chemiluminescent, but is preferably colorimetric in nature (for ease of use). The test device for determining concentration levels of both H-hCG and hCG (which may include intact hCG and H-hCG or both intact hCG and H-hCG as well free β-subunit of hCG and H-hCG) is preferably a nitrocellulose-based (or other appropriate polymeric material) calorimetric sandwich assay (nitrocellulose-based sandwich assay) or two antibody test based upon a capture antibody and a detection antibody (at least one) wherein the capture and detection antibodies recognize and bind different epitopes of hCG and hyperglycosylated hCG, and wherein one of the antibodies (the detection antibody) is coupled to a label that produces a detectable or colorimetric signal (through a dye such as a gold-based dye) and the other antibody, the capture antibody, is anchored to a support, preferably a polymeric material, preferably a nitrocellulose or other film layer, wherein the capture antibody is fixed in a line in the film layer. In this preferred assay, both the capture antibody and the detection antibody are specific for different epitopes on both hCG and H-hCG, such that both hCG and hyperglycosylated hCG may be measured. In many instances, depending upon the antibodies chosen, either intact hCG and intact H-hCG may be measured. In preferred aspects, the capture antibody is specific for the β-subunit of intact hCG and H-hCG as well as the free subunit of hCG and H-hCG and the detection antibody may be much less specific provided that the antibody binds, and consequently labels, essentially all of the hCG and H-hCG (intact and/or subunit) which is bound to the capture antibody. In this assay the capture antibody is specific for a different epitope on both hCG and H-hCG than is the detection antibody although both the capture and detection antibodies (in the case of the detection antibody, either singly or collectively) are specific for both hCG and H-hCG and are calibrated to bind substantially equally to the two antigens to maximize accuracy to at least about 95%, preferably at least about 98%, preferably at least about 99% and preferably at least about 100%. This is an unexpectedly accurate method for testing pregnancy in woman from about the fourth week after the woman's previous menstrual cycle to the 13^{th} week or beyond.

In this point of care diagnostic test, the detection antibody is linked to dye (gold-based or other) and initially is in an upper layer material which is porous to liquid and is free to move to a lower layer when it comes into contact with liquid, such as blood, serum, plasma or urine, which contains hCG and H-hCG. The detection antibody of the upper layer is free to move from the upper layer (preferably a porous sponge material) to the lower layer. The other antibody (the capture antibody) is anchored in the nitrocellose matrix or other similar material in a shape like a line. The hCG and H-hCG in serum would enter the device and bind to the antibody-dye. There is an opening in the device case called the “result window”, exposing any color from the dye. The hCG and H-hCG antibody-dye would then move into or through the nitrocellulose matrix until it reaches the anchored captured antibody. The result (“result line”) would be a colored line in the “result window”. A further line of die would be shown in the “result window”. This is the “control dye line” or line generated by a dye that corresponds to the color and intesity that would be observed in the line in the “result window” from the antibody-dye: hCG and hyperglycosylated hCG: anchored antibody sandwich if blood/serum/urine concentration was formed by at least about 6 mIU/ml (corresponding to about 0.55 ng/ml of hCG and H-hCG). If the “result line” was not as intense as the “control dye line” then hCG and H-hCG levels of <0.55 ng/ml are present indicating that the patient was not pregnant. If the “result line” was similar or more intense than the “control dye line” then the patient is predicted to be pregnant with a high 95% accuracy. The aim would be for a test of sensitivity calibrated preferably to a combined hCG and H-hCG level of about 0.55 g/ml.

Two preferred devices are available in this point of care/home pregnancy test assay. In the first methodology, antibody concentrations are adjusted such that the test only shows a positive result at a hCG and H-hCG serum concentration of 0.55 ng/ml or greater. In this case, a positive result would mean that the patient is pregnant. A second, more sophisticated device/methodology would have a control line. In this aspect, a positive result is observed and is compared in intensity to a second line, or a control line, that corresponds to the exact intensity of line given by 0.55 ng/ml. If the resulting test line is equal to, more distinct or darker than the control line, this would evidence the existence of pregnancy. If the result line is less distinct or lighter than the control line it would evidence that the patient is not pregnant. Confirmation of pregnancy through ultrasound or other methodology could confirm the results.

The following examples are presented to illustrate assays and methods used for detecting pregnancy. The methodology and results may vary depending on the parameters of the assays being used, as well as the antigens being screened. The examples are not intended in any way to otherwise limit the scope of the invention.

### HCG/Hyperglycosylated hCG Assay

A new assay is developed to equally detect regular intact hCG and intact H-hCG (in this example, not the free B form). The assay is a microtiter plate enzyme-immunometric assay measured in a 96 well microtiter plate reader. The capture antibody used is monoclonal 2119 (from Unipath Inc., London UK). This antibody is an anti-alpha subunit antibody which binds both hCG and hyperglycosylated hCG. Plating a 96 well microtiter plate requires 20 ml (0.2 ml per well). The tracer antibody used was peroxidase labeled monoclonal 4001 (4001-POD), purchased ladled from the Medix division of Genzyme. The assay 96 well assay also requires 20 ml of tracer (0.2 ml/well). The 4001-POD is specific for the core or 3D area of the β-subunit (not the C-terminal peptide). The 2119 antibody was at a concentration of 3.5 mg/ml and the 4001-POD was at 1.0 mg/ml. The antibodies were then calibrated in order to ensure equal reactivity to both hCG and hyperglycosylated hCG.

We examined the 2119 monoclonal at plating concentrations of 1:100, 1:200, 1:400. 1:800, 1:1600 and 1:3200 dilution or 0.035, 0.018, 0.009 0.004, 0.002, 0.001 mg/ml. Each of the 6 plating concentrations was matched with 6 dilutions of the 4001-POD, 1:100, 1:200, 1:400. 1:800, 1:1600 and 1:3200 dilution or 0.01, 0.005, 0.0025 0.0012, 0.0006, 0.0003 mg/ml. This is 36 combinations total. A further parameter was incubation time. Results were tested at 0.5 hour, 1 hour, 2 hour and 4 hour. This made the total number of combinations tested 144. Each combination was evaluated with a standard curve of pure regular hCG (NIH standard CR127) and pure hyperglycosylated (JEG-3 standard from Nichols Institute Diagnostics). The 2 standard curves were covering the desired sensitivity, 0, 0.2, 0.5, 1, 2, 5 and 10 ng/ml.

The following 3 criterion were used to choose the best antibody concentrations and incubation time—

1. Background, or result with 0 ng/ml standard

2. Range of values, slope of spectrometric result (from enzyme-labeled reaction) with change in concentration, the steeper the result the more accurate the test.

3. Ability to give similar or preferably, identical spectrometric results with different concentration of hCG as with H-hCG. In the event that this cannot be achieved, while still satisfying criterion 1 and 2, then antibodies are inappropriate and a change of antibodies is indicated.

The optimal results under all 3 criterion were achieved with 1:400 2119 capture antibody and 1:200 4001-POD tracer.

The assay is now calibrated with 0.31, 0.62, 1.25, 2.5, 5 and 10 ng/ml (with 0 ng/ml blank subtracted) regular hCG standards (pure recombinant technology hCG standard, has 0 H-hCG.

Using this assay with this standard curve we evaluated detection of JEG-3H-hCG. The results were 0.028, 0.59, 1.27, 2.6, 5.0 and 9.8 ng/ml (with 0 ng/ml blank subtracted) respectively. This is almost a perfect match or equal detection. We plotted the two sets of results against each other, the r2, or correlation coefficient was 0.9985, an extremely high degree of accuracy.

TABLE 1

| Week of Pregnancy | | ITA |
|---|---|---|
| (from last menstrual cycle) | hCG conc. | as % of HCG + ITA |
| 3^{rd} | 3% | 97% |
| 4^{th} | 28% | 72% |
| 5^{th} | 50% | 50% |
| 6^{th} | 74% | 26% |
| 7^{th} | at least 90% | no more than 10% |
| 8^{th} and later | more than 90% | less than 10% |

References for Table 1 (11, 13, 14, 23)

Various publications and/or references have been cited herein, the contents of which are incorporated herein by reference.

While this invention has been described with respect to various specific examples and embodiments, it is to be understood that the invention is not limited thereto and that it can be variously practiced with the scope of the following claims.

### REFERENCES

1. Cole L A. Immunoassay of human chorionic gonadotropin, its free subunits, and metabolites. Clin Chem 1997;43:2233-43.
2. Cole L A, Kardana A. Discordant results in human chorionic gonadotropin assays. Clin Chem 1992;38:263-70.
3. Hussa R O. Immunologic and physical characterization of human chorionic gonadotropin and its subunits in cultures of human malignant trophoblast. J Clin Endocrinol Metab 1977;44:1154-62.
4. Mann K, Karl H J. Molecular heterogeneity of human chorionic gonadotropin and its subunits in testicular cancer. Cancer 1983;52:654-60.
5. Cole L A, Hussa R O. The carbohydrate on human chorionic gonadotropin produced by cancer cells. Adv Exp Med Biol 1984; 176:245-70.
6. Imamura S, Armstrong G A, Birken S, Cole L A, Canfield R E. Detection of desialylated forms of human chorionic gonadotropin. Clin Chim Acta 1987;163:339-49.
7. Cole L A. The O-linked oligosaccharides are strikingly different on pregnancy and choriocarcinoma hCG. J Clin Endocrinol Metab 1987;65:811-13.
8. Cole L A. O-Glycosylation of proteins in the normal and neoplastic trophoblast. Troph Res 1987;2:139-48.
9. Amano J, Nishimura R, Mochizuki M, Kobata A. Comparative study of the mucin-type sugar chains of human chorionic gonadotropin present in the urine of patients with trophoblastic diseases and healthy pregnant women. J Biol Chem 1988;263: 1157-65.
10. Elliott M M, Kardana A, Lustbader J W, Cole L A. Carbohydrate and peptide structure of the a- and b-subunits of human chorionic gonadotropin from normal and aberrant pregnancy and choriocarcinoma. Endocrine 1997;7:15-32.
11. Cole L A, Shahabi S, Oz U A, Bahado-Singh R O, Mahoney M J. Hyperglycosylated human chorionic gonadotropin (invasive trophoblast antigen) immunoassay: A new basis for gestational Down syndrome screening. Clin Chem 1999;45:2109-19.
12. Birken S, Krichevsky A, O'Connor J, Schlatterer J, Cole L A, Kardana A, Canfield R. Development and characterization of antibodies to a nicked and hyperglycosylated form of hCG from a choriocarcinoma patient: generation of antibodies that differentiate between pregnancy hCG and choriocarcinoma hCG. Endocrine 1999;10:137-44.
13. O'Connor J F, Ellish N, Kakuma T, Schlatterer J, Kovalevskaya G. Differential urinary gonadotrophin profiles in early pregnancy and early pregnancy loss. Prenat Diagn 1998;18:1232-40.
14. Kovalesvskaya G, Birken S, Kakuma T, O'Connor J F. Early pregnancy human chorionic gonadotropin (hCG) isoforms measured by immunometric assay for choriocarcinoma-like hCG. J Endocrinol 1999;161:99-106.
15. Kovaleskaya G, Genbacev O, Fisher S J, Caceres E, O'Connor J F. Trophoblast origin of hCG isoforms: cytotrophoblasts are the primary source or choriocarcinoma-like hCG. Mol Cell Endocrinol 2002;94:147-55.
16. Butler S A, Khanlian S A, Cole L A. Detection of early pregnancy forms of human chorionic gonadotropin by home pregnancy test devices. Clin Chem 2001;47:2131-6.
17. Lei Z M, Taylor D D, Gercel-Taylor C, Rao C V. Human chorionic gonadotropin promotes tumorigenesis of choriocarcinoma JAR cells. Troph Res 1999; 13:147-59.
18. Cole L A, Shahabi S, Butler S A, Mitchell H, Newlands E S, Behrman H R, Verrill H L. Utility of commonly used commercial human chorionic gonadotropin immunoassays in the diagnosis and management of trophoblastic diseases. Clin Chem 2001;47:308-15.
19. Wilcox A J, Weinberg C R, O'Connor J F, Baird D D, Schlatterer J P, Canfield R E, Armstrong E G, Nisula B C. Incidence of early loss of pregnancy. N Engl J Med 1988;319:189-94.
20. Bjercke S, Tanbo T, Dale P O, Morkrid L, Abyholm T. Human chorionic gonadotrophin concentrations in early pregnancy after in-vitro fertilization. Hum Reprod 1999;14:1642-6.
21. Birken S, Berger P, Bidart J M, Weber M, Bristow A, Norman R, Sturgeon C, Stenman U H. Preparation and characterization of new WHO reference reagents for human chorionic gonadotropin and metabolites. Clin Chem 2003;49: 144-54.
22. Shahabi S, Oz U A, Bahado-Singh R O, Mahoney M J, Omrani A, Baumgarten A, Cole L A. Serum hyperglycosylated hCG: a potential screening test for fetal Down's syndrome. Prenat Diagn 1999; 19:488-89.
23. Cole L A, Khanlian S A, Sutton J M, Davies S, Stephens N. Hyperglycosylated hCG (Invasive Trophoblast Antigen, ITA) a Key Antigen for Early Pregnancy Detection. Clin Biochem, 2003;36;647-655

1. A method for detecting pregnancy in a woman comprising:
a. contacting a biological sample of a woman with at least one antibody which is calibrated to specifically bind both hCG and hyperglycosylated hCG (H-hCG) substantially equally, and wherein said antibody is coupled to a label effective to produce a signal; and
b. measuring the concentration of hCG and H-hCG in said sample by detecting a signal produced by said label, wherein the presence of a detectable signal indicates pregnancy in the woman.

2. The method of claim 1, wherein the biological sample is a urine or serum sample.

3. The method of claim 1, wherein the label is an acridinium ester or a dye.

4. The method of claim 1, wherein the label is a dye.

5. The method of claim 1 wherein said hCG and hyperglycosylated hCG is intact hCG and intact hyperglycosylated hCG.

6. The method according to claim 1 wherein said concentration of hyperglycosylated hCG is measured using a calorimetric or chemiluminescent assay.

7. The method according to claim 6 wherein said assay is a nitrocellulose-based sandwich assay which utilizes a single capture antibody and at least one detection antibody.

8. The method according to claim 6 wherein said concentration of hCG and H-hCG is at least about about 6 miu/mL.

9. A method for detecting pregnancy in a woman to be tested for pregnancy comprising:
a. providing at least one antibody which specifically binds to both hCG and H-hCG and wherein said antibody is coupled to a label;
b. calibrating said antibody at a concentration and incubation time so that said antibody binds both hCG and H-hCG substantially equally after a set time;
c. obtaining a biological sample from said woman to be tested and contacting said sample with said antibody; and
d. measuring the concentration of hCG and H-hCG in said sample by detecting a signal produced by said label after said set time, wherein the presence of a detectable signal above a predetermined value indicates pregnancy in the woman.

10. The method of claim 9, wherein the biological sample is a urine or serum sample.

11. The method of claim 9, wherein the label is an acridinium ester or a dye.

12. The method of claim 9, wherein the label is a dye.

13. The method of claim 9, wherein the label is an enzyme which reacts with a compound to produce a chemiluminescent signal from the reaction of said enzyme with said compound.

14. The method of claim 9 wherein said hCG and H-hCG is intact hCG and intact H-hCG.

15. The method according to claims 10 wherein said concentration of H-hCG is measured using a colorimetric or chemiluminescent assay.

16. The method according to claim 15 wherein said assay utilizes a single capture antibody and at least one detection antibody.

17. The method according to claim 16 wherein said assay is a nitrocellulose-based sandwich assay.

18. A method for detecting pregnancy in a woman comprising:
a. contacting a biological sample of a woman with at least one antibody which is calibrated to specifically bind hCG, H-hCG, and the free β-subunits of hCG and H-hCG substantially equally, and wherein said antibody is coupled to a label effective to produce a signal; and
b. measuring the concentration of hCG, H-hCG and the free β-subunits of hCG and hyperglycosylated hCG in said sample by detecting a signal produced by said label, wherein the presence of a detectable signal indicates pregnancy in the woman.

19. The method of claim 18, wherein the biological sample is a urine or serum sample.

20. The method of claim 18, wherein the label is an acridinium ester or a dye.

21. The method of claim 19, wherein the label is a dye.

22. The method according to claim 18 wherein said concentration of hCG, H-hCG and the free β-subunits of hCG and H-hCG is measured using a colorimetric or chemiluminescent assay.

23. The method according to claim 19 wherein said assay utilizes a single capture antibody and at least one detection antibody.

24. The method according to any of claims 23 wherein said assay is a nitrocellulose-based sandwich assay.

25. The method according to claim 24 wherein said concentration is about 2 miu/mL.

26. A method for detecting pregnancy in a woman comprising:
a. contacting a biological sample of a woman with at least one antibody which is calibrated to specifically bind the free β-subunits of hCG and H-hCG substantially equally, and wherein said antibody is coupled to a label effective to produce a signal; and
b. measuring the concentration of the free β-subunits of hCG and H-hCG in said sample by detecting a signal produced by said label, wherein the presence of a detectable signal indicates pregnancy in the woman.

27. A method for detecting pregnancy in a woman comprising:
a. providing at least one antibody which specifically binds to hCG, H-hCG and the free β-subunits of hCG and H-hCG and wherein said antibody is coupled to a label;
b. calibrating said antibody at a concentration and incubation time so that said antibody binds hCG, H-hCG and the free β-subunits of hCG and H-hCG substantially equally;
c. obtaining a biological sample of a woman and contacting said sample with said antibody; and
d. measuring the concentration of hCG, H-hCG and the free β-subunits of hCG and H-hCG in said sample by detecting a signal produced by said label, wherein the presence of a signal detecting said concentration above a predetermined value indicates pregnancy in the woman.

28. The method of claim 27, wherein the biological sample is a urine or serum sample.

29. The method of claim 27, wherein the label is an acridinium ester or a dye.

30. The method of claim 27, wherein the label is a dye.

31. The method of claim 27, wherein the label is an enzyme which reacts with a compound to produce a chemiluminescent signal from the reaction of said enzyme with said compound.

32. The method according to claim 27 wherein said concentration of hCG, H-hCG and the free β-subunits of hCG and H-hCG is measured using a calorimetric or chemiluminescent assay.

33. The method according to claim 30 wherein said assay is a nitrocellulose-based sandwich assay which utilizes a single capture antibody and at least one detection antibody.

34. The method according to claim 31 wherein said assay is a nitrocellulose-based sandwich assay.

35. The method according to claim 34 wherein said concentration (step d) is about 6 miu/mL.
